(19) United States
(12) Patent Application Publication
(10) Pub. No.: US 2004/0123644 A1
(43) Pub. Date: Jul. 1, 2004

(54) Device for determining the condition of oil

(76) Inventors: Bernhard Jakoby, Wien (AT); Christian Krummel, Kirchentellinsfurt (DE)

Correspondence Address: KENYON & KENYON, ONE BROADWAY, NEW YORK, NY, 10004, US

(21) Appl. No.: 10/666,224

(22) Filed: Sep. 18, 2003

(30) Foreign Application Priority Data
Sep. 19, 2002 (DE) 102 43 510.3

(51) Int. Cl. G01N 33/30

(52) U.S. Cl. 73/19.11

(57) ABSTRACT

A device for determining the concentration of volatile constituents of motor oil is described.

### FIELD OF THE INVENTION

The present invention is based on a device for determining the condition of oil.

### BACKGROUND INFORMATION

Analysis methods are known for analyzing the ageing of lubricants in combustion engines, e.g., in motor oil, which may be carried out in a laboratory. To that end, a number of physical and physical-chemical measuring methods are used which in each case evaluate one specific ageing aspect of the oil sample being examined. It is disadvantageous that such laboratory analyses cannot be carried out in vehicles while driving, which would permit a continuous monitoring of the condition of the lubricants.

### SUMMARY

In accordance with an example embodiment of the present invention, it is possible to evaluate the condition of the oil onboard, since the oil condition may be continuously monitored. For example, no sampling is necessary for measuring the state of the oil. Conventional sensors are based predominately on simple physical measuring principles such as the measurement of the polarizability (permittivity) of the lubricant to be examined, i.e., particularly of the oil to be examined, or the measurement of the electric conductivity of the oil. However, the knowledge of these variables alone is not adequate for a reliable evaluation of the oil condition.

Of the numerous parameters utilized during laboratory analyses for reliable information about the condition of the oil, the oil viscosity and the acid content or basicity of the oil are two of the most important features. Methods which allow an onboard determination of the acid content of motor oil are not known at present. The acid content of motor oil is usually characterized using the total base number (TBN) and the total acid number (TAN). The fact that the acid and basic constituents in motor oil are, primarily, not present in dissolved form, additionally hinders the characterization of this oil property. In contrast to aqueous solutions, whose acid content may easily be indicated using the pH value, the conditions are more difficult to determine for oils. As an alternative to the usual titration methods for determining the acid content of motor oil in the liquid phase, for which a sampling is inevitably necessary, when using the example device of the present invention, volatile compounds such as acetaldehyde, acetone, acetic acid or benzaldehyde are detected, whose concentration is correlated in general with the total base number or the total acid number, or at least with the ageing condition of the oil. In particular, according to the present invention, a device for determining the condition of oil is provided which makes do without sampling and which may be produced easily and cost-effectively, and thus may be incorporated in a motor vehicle in operation. That the measurement of the indicated volatile compounds is indeed possible in principle in the laboratory, e.g., with the aid of gas chromatography or mass spectroscopy, changes nothing in the fact that this is not possible for the practical use of determining the condition of oil during the running operation of a motor vehicle or a combustion engine, but rather may only be carried out using bulky and complex measuring devices. In accordance with the present invention, the volatile gas constituents of a liquid to be examined is detected, i.e., particularly a motor oil, using a compact gas sensor which, according to the present invention, is provided in particular as a semiconductor gas sensor based on tin oxide.

It is particularly advantageous that the device is usable onboard in a motor vehicle. Therefore, the motor oil may be monitored long-term, giving rise to the positive concomitants such as that of an oil change only when necessary and the like. It is also advantageous that the concentration of volatile constituents is a function of the total base number of the oil. Therefore, from the measurement of the concentration of the volatile constituents, it is possible on one hand to infer the total base number, and in a further step, to infer the condition of the motor oil. It is also advantageous if the device includes a semiconductor gas sensor having a sensitive layer, the sensitive layer including metal oxides. It is thereby possible to provide the semiconductor gas sensor in a particularly cost-effective manner and nevertheless in a manner that it is sufficiently sensitive for the volatile gas constituents of the motor oil to be determined. Furthermore, it is advantageous if the sensitive layer includes metal oxides of the metals Sn, W, Zn, Fe, Mo and/or Cr with admixtures of less than 1% of metals and/or metal oxides from the group of metals Co, Ni, Mo, Re, Zn, Cr, Al, Ce and/or Mn, and with admixtures of less than 1% of metals from the group of metals Ag, Au, Pt and/or Pd. It is thereby possible, by a different fashioning of the sensitive layer, to either especially optimize it for different situations, or to provide a widely usable sensitive layer having a sufficient sensitivity for many measuring situations, which means a sensitive layer of this type may be produced particularly inexpensively. It is furthermore advantageous if the device has an SAW (surface acoustic waves) sensor or a BAW (bulk acoustic waves) sensor or a chemiluminescence sensor. It is thereby advantageously possible to use alternative sensor principles for the sensor of the present invention, i.e., for the example device of the present invention. Another advantage is that the example device may have a first membrane which is impermeable for oil, but is permeable for the volatile constituents. It is thereby possible, using simple means, to employ a gas sensor according to the present invention for determining the condition of oil. Moreover, it is advantageous if the volatile constituents are acetaldehyde, acetone, acetic acid and/or benzaldehyde. This permits particularly easy detectability using the sensitive layer according to the present invention. Moreover, it is advantageous if the device includes a gas compartment which is separated from an oil-containing region by the first membrane. It is thereby possible to keep the device particularly stable over its service life, because the gas-sensitive layer is not soiled or impaired by contact with the oil. It is also advantageous that the first membrane is moistened by oil.

### BRIEF DESCRIPTION OF THE DRAWINGS

An example embodiment of a device according to the present invention is explained in detail in the following description and is shown in the drawings.

FIG. 1 shows an example device of the present invention with its environment according to the present invention.

FIG. 2 shows an example gas sensor according to the present invention.

### DETAILED DESCRIPTION OF EXAMPLE EMBODIMENTS

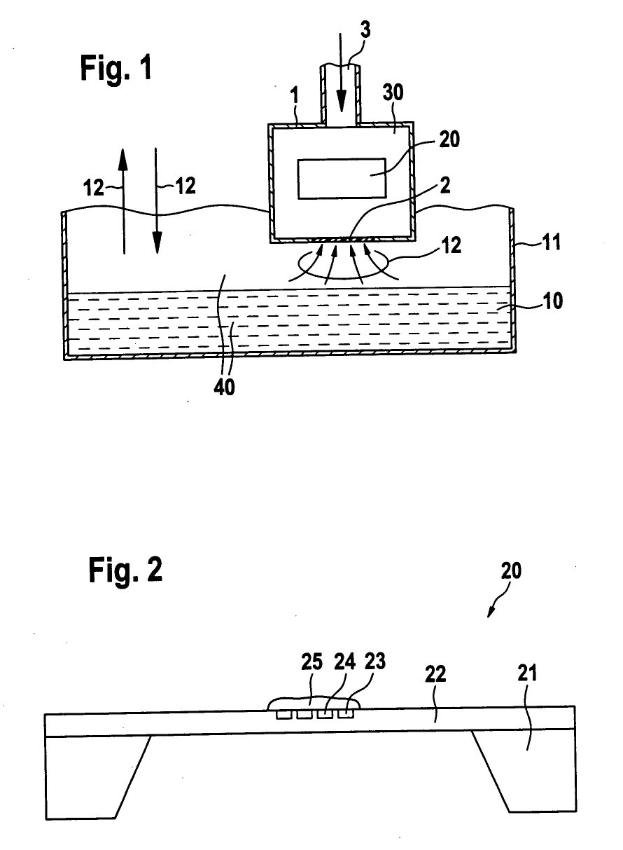

FIG. 1 shows a configuration in principle of device 1 according to an example embodiment of the present invention. Device 1 includes a gas sensor 20 which, according to the present invention, is provided in particular as a semiconductor gas sensor, and therefore is also designated in the following as semiconductor gas sensor 20. Gas sensor 20 is shown in greater detail in FIG. 2. Moreover, device 1 includes a gas compartment 30 in which gas sensor 20 is located.

In device 1 according to the present invention, gas compartment 30 is separated from an oil-containing region 40 by a first membrane 2. First membrane 2 is provided as a so-called oil-repellent membrane. This means that although first membrane 2 is permeable for volatile constituents, which are provided in FIG. 1 with reference numeral 12 and which are present in oil-containing region 40, first membrane 2 is impermeable for oil, which is provided in FIG. 1 with reference numeral 10 in an oil reservoir 11.

Oil reservoir 11 is provided in particular as oil pan 11 of a combustion engine. With the aid of connections (not shown) to oil pan 11, an oil circulation is indicated in FIG. 1, designated by reference numeral 12 and two arrows. Generally, the oil is circulated through oil circulation 12 by an oil pump (not shown), oil circulation 12 usually also having an oil filter (not shown). In oil reservoir 11, oil 10 forms in particular a liquid phase and, above the liquid phase, a region in which, according to the present invention, for example, oil squirts are provided. Device 1 of the present invention is located either, as shown in FIG. 1, above the liquid phase of oil 10, or else in direct contact with the oil, that is to say, the liquid phase of oil 10 moistens first membrane 2. As already mentioned, first membrane 2 is provided as a gas-permeable layer which, however, is not permeable for oil 10. According to the present invention, first membrane 2 is provided in particular as an oil-repellent Teflon membrane. Through it, one or more of the volatile indicator substances such as acetaldehyde, acetone, acetic acid, benzaldehyde, which, as volatile constituents in the form of indicator substances, are characteristic for the ageing of the oil, is/are able to get into gas compartment 30 of device 1, where gas sensor 20, likewise disposed in gas compartment 30, is able to measure the concentration of this/these indicator substance(s).

Gas sensor 20 is protected from oil 10 by first membrane 2. According to the present invention, gas compartment 30 of device 1 should have a defined admission possibility for air. This is represented by an arrow and reference numeral 3 in FIG. 1. This may be necessary in the present invention because the burning of the indicator substances when using a semiconductor gas sensor usually consumes oxygen. When using a gas sensor 20 in the form of an SAW (surface acoustic waves) sensor or a BAW (bulk acoustic waves) sensor or a chemiluminescence sensor, according to the present invention, it is not necessarily requisite to provide an admission possibility for air, because such types of sensors are based on sensor principles which do not consume oxygen. If, when using these last-named sensor principles, no admission possibility 3 for air is provided, a concentration equilibrium of the substances to be detected is established in gas compartment 30.

In a modification of device 1 of the present invention shown in FIG. 1, first membrane 2 may also be in direct contact with oil 10, i.e., with its liquid phase.

According to the present invention, gas sensor 20 may be implemented by various technologies. For reasons of cost, semiconductor gas sensors 20 are preferably used in the present invention. Such a semiconductor gas sensor 20 is shown in FIG. 2. It includes a substrate 21, a second membrane 22 and a sensitive layer 25. Sensitive layer 25 interacts with indicator substances 12, which get into gas compartment 30 through first 30 membrane 2. Sensitive layer 25 includes, in particular, powdery metal oxides which are sintered by a burning process. According to the present invention, gas sensor 20 has a heating structure 23 and an electrode structure 24. Heating structure 23 heats sensitive layer 25 to an elevated temperature of, for instance, 100 to 400° C. according to the present invention. In response to the presence of the gases to be detected or the indicator substances to be detected, one electrically measurable property of sensitive layer 25 of gas sensor 20 changes. The resistance, the impedance or the capacitance of sensitive layer 25 may be used in particular here as the electrically measurable property. These electrically measurable properties are measured via electrode structure 24. Electrode structure 24 is connected to contact structures (not shown) on semiconductor sensor 20. Electrical signals from electrode structure 24 may thus be routed to an evaluation unit, not shown in FIG. 2.

According to the present invention, sensitive layer 25 is made of metal oxides of the metals Sn, W, Zn, Fe, Mo or Co. In this context, sensitive layer 25 includes, in particular, admixtures of less than one percent of metals or metal oxides of the group of metals Cu, Ni, Mo, Re, Zn, Cr, Al, Ce, Mn, and further admixtures of likewise less than one percent of noble metals from the group of metals Ag, Au, Pt or Pd. In this connection, according to the present invention, the admixtures have, in particular, a minimum portion of 0.0001%. The base material of sensitive layer 25 and the admixtures are especially selected according to the present invention so that the signal, i.e., the electrical property to be measured, such as the change in resistance of sensitive layer 25, of the gas sensor is at a maximum.

What is claimed is:

1. A device for determining the condition of motor oil, comprising:
an arrangement configured to determine a concentration of volatile constituents of the oil.

2. The device as recited in claim 1, wherein the device is configured for use onboard in a motor vehicle.

3. The device as recited in claim 1, wherein the concentration of volatile constituents is a function of the total base number of the oil.

4. The device as recited in claim 1, wherein the arrangement includes a semiconductor gas sensor having a sensitive layer, the sensitive layer including metal oxides.

5. The device as recited in claim 4, wherein the sensitive layer includes metal oxides of at least one of Sn, W, Zn, Fe, Mo, and Cr, with admixtures of less than one percent of at least one of metals and metal oxides, selected from a group of metals consisting of Cu, Ni, Mo, Re, Zn, Cr, Al, Ce and Mn, and with admixtures of less than one percent of metals selected from a group of metals consisting of Ag, Au, Pt, and Pd.

6. The device as recited in claim 1, wherein the arrangement includes one of a surface acoustic wave (SAW) sensor, a bulk acoustic wave (BAW) sensor, or a chemiluminescence sensor.

7. The device as recited in claim 1, wherein the arrangement includes a first membrane which is impermeable for oil, but is permeable for the volatile constituents.

8. The device as recited in claim 1, wherein the volatile constituents include at least one of acetaldehyde, acetone, acetic acid, and benzaldehyde.

9. The device as recited in claim 7, wherein the arrangement has a gas compartment that is separated from an oil-containing region by the first membrane.

10. The device as recited in claim 7, wherein the first membrane is moistened by the oil.
